(19) United States
(12) Patent Application Publication
(10) Pub. No.: US 2025/0352380 A1
(43) Pub. Date: Nov. 20, 2025

(54) Wearable device to reduce pain and promote healing of lower back problems

(71) Applicant: Leona E LLC

(72) Inventor: Paul Sacco, Windsor, CO (US)

(21) Appl. No.: 18/389,324

(22) Filed: Nov. 14, 2023

Related U.S. Application Data
(63) Continuation-in-part of application No. 16/938,391, filed on Jul. 24, 2020, now Pat. No. 11,813,187.
Application No. 16/938,391 is a continuation of application No. 15/788,191, filed on Oct. 19, 2017, now abandoned.

(51) Int. Cl. A61F 5/02; A61F 5/04; A61F 5/042

(52) U.S. Cl. CPC A61F 5/028; A61F 5/02; A61F 5/024; A61F 5/042; A61F 5/04

(57) ABSTRACT

A device that decompresses the lower spine while the wearer is seated or standing. The device includes two belt-shaped straps. The first strap circles the upper torso just below the breast line. The second strap circles the waist at or slightly above the normal belt line riding on the hip. In the present invention, the lower strap can be a normal belt. The straps can be equipped with pockets that are aligned along the upper edge of the upper strap and the lower edge of the lower strap designed to receive and hold flat splints. Typically, two rigid flat splints extend vertically between the straps centered laterally on the left and right sides of the torso. When the device is worn, it decompresses the lower spine when the person stands or sits. In the new embodiments the flat splints are adjustable length and the pockets are horizontally moveable along the lower and upper straps and the “U”shaped end that attaches to the wearers belt is optional.

This is a continuation in part of application Ser. No. 16/938,391 filed Jul. 24, 2020 which was a continuation of application Ser. No. 15/788,191 filed Oct. 19, 2017. applications Ser. Nos. 16/938,391 and 15/788,191 are hereby incorporated by reference in their entireties.

### BACKGROUND

### Field of the Invention

The present invention relates to devices for back problems and more particularly to a wearable device that dramatically reduces pain and promotes healing of Spinal Stenosis, Degenerative Disc Disease, Herniated Discs, Muscle Spasms, and Sciatica.

### Description of the Problem Solved and of the Prior Art

Low back pain is the single leading cause of disability worldwide. According to the Global Burden of Disease 2010, an average of 30 million adults experience lower back pain at some point in their lifetimes, and it is the most common cause of job-related disability. Spinal Degenerative Joint Disease describes a number of lower back conditions that primarily affect people when they become older. Spondylosis is a term that refers to the general degeneration of the spine associated with normal wear and tear that occurs in the joints, discs, and bones of the spine as people get older. One of the many mechanical causes of low back pain is a condition called Spinal Stenosis where the spinal canal begins to narrow causing the open spaces between the vertebrae to decrease in size. The tightness can pinch the spinal cord and/or Sciatic Nerve causing pain, tingling, or numbness in the legs, arms, or torso. Common treatments include medications and physical therapy. If the pain cannot be controlled this way, surgery may be recommended. Unfortunately, surgery does not always work. Some spine decompression techniques include ice and even hanging a patient upside-down to relieve the pressure. While hanging upside-down is effective, it is hardly practical as a long-term cure and can even cause strokes if the body remains at more than a 30-degree angle too long.

It has been shown that if the lower back can be decompressed for long periods of time, not only is pain relieved, but also actual healing can take place. Healing cannot occur when the spine is in a compressed state. While extreme techniques like hanging upside down, or engaging in expensive decompression sessions on a specialized mechanical bed at the doctor's office, can result in the desired decompression, these alternatives are simply not practical and can even be dangerous.

U.S. Pat. No. 4,708,130 by Grudem shows an immobilization device that is typically used after back surgery. This device has two splints that are forward of the side of the user's body and a solid back brace member that resides in the center of the back. This device immobilizes the back, but fails to provide decompression while sitting. The forward position of the splints also gets in the way of normal movements and can actually cause pain in the chest when the user tries to bend forward.

U.S. Pat. No. 3,420,230 by Ballard teaches a device with two straps and a single back splint. This device fails to provide decompression when seated or bending forward.

It would be particularly advantageous to have a device that decompressed the lower spine while the user is simply sitting, since sitting is the most comfortable position and is a position that many people must assume at work or leisure for long hours anyway.

### SUMMARY OF THE INVENTION

The present invention relates to a device that decompresses the lower spine while the wearer is seated. The device includes two (and in some cases, only one) belt-shaped straps. The first strap circles the upper torso just below the breast line. It can thus be used by women as well as men without putting undue pressure on the breasts. The second strap circles the waist at the normal belt line and rides on the hips. In some embodiments of the invention only one strap is used with the lower strap being a normal belt. The straps are typically equipped with a series of pockets that are aligned along the upper edge of the upper strap and the lower edge of the lower strap that are designed to receive and hold flat splints. In alternate embodiments, the splints can be permanently attached with no pockets. Typically, two rigid flat splints extend vertically between the upper and lower straps on the left and right sides of the torso. With the straps and splints correctly positioned, the device decompresses the lower spine when the user sits and slightly leans forward, or leans from side to side. It maintains maximum decompression as long as the person remains seated. The device, normally worn under the clothes, does not interfere with clothing, movement, work, or appearance. Since many people remain seated for long periods each day, the present invention maintains a state of spinal decompression during these long periods. Decompression, in addition to relieving pain, improves posture and allows blood, hydration and nutrients to flow into the problem area and enhance the body's natural healing response. This simply cannot happen when the area is being continually pinched by the natural forces of gravity, much less by other mechanical physical conditions of the spine.

The present invention grips the upper back and hips and performs an expansive motion when the wearer gently leans forward or side to side, expanding the lower spine by simply sitting in a chair. It also creates a gentle expansion while the wearer is standing and engaging in daily activities and sports. It is worn against the skin for best grip and can be comfortably worn on a daily basis for hours during the day under clothing while sitting at a desk or keyboard, or while standing and moving. As a side effect, the present invention helps correct the wearer's posture by making the user sit up straight while simultaneously decompressing the lower spine helping to alleviate pain.

### DESCRIPTION OF THE FIGURES

Attention is now directed to several drawings that illustrate features of the present invention. FIGS. 1-9 show the present, patented invention. FIGS. 10-19 show the new matter which is the subject of this CIP.

FIG. 1 shows an embodiment of the present invention being worn (outside clothing for clarity).

FIG. 2 present invention shows the embodiment of FIG. 1 assembled, but not being worn.

FIG. 3 present invention shows an exploded view of the device of FIG. 2.

FIG. 4 present invention shows an isometric view of one of the straps showing an arrangement of pockets.

FIG. 5 present invention shows a bottom-up view of the strap of FIG. 4.

FIG. 6 present invention shows an isometric view of an assembled version of the embodiment of FIGS. 4-5.

FIG. 7 present invention shows top and side views of various length splints.

FIG. 8 present invention shows a front and back view of a typical strap.

FIG. 9 present invention shows an alternate embodiment of the invention worn with only one strap and a standard belt.

FIG. 10 shows new matter, an improvement of the present invention, a moveable pocket assembly that can be moved horizontally along the strap.

FIG. 11 shows new matter, the outside of half of a sliding device. which fits with the pocket assembly to assist users of tall or short heights.

FIG. 12 shows new matter, the reverse side of the same half of the sliding device of FIG. 11.

FIG. 13 shows new matter, a rail piece, into which the sliding device of FIGS. 11 and 12 fits.

FIG. 14 shows new matter, the reverse side of the FIG. 13 rail piece.

FIG. 15 shows new matter, an initial setup connection of the hook loop strap from the sliding device of FIG. 11 to the rail piece of FIG. 13.

FIG. 16 shows new matter, the position of the completed sliding splint mechanism of FIG. 15 for a taller person.

FIG. 17 shows new matter, the position of the completed sliding splint mechanism of FIG. 15 for a shorter person.

FIG. 18 shows new matter, isometric views of the sliding splint mechanism attached to movable pockets for upper and lower supports adjusted for a taller person.

FIG. 19 shows new matter, isometric views of the sliding splint mechanism attached to movable pockets for upper and lower supports adjusted for a shorter person.

### DESCRIPTION OF THE PREFERRED EMBODIMENTS

The present invention relates to a device that can be worn under clothing to decompress the lower spine relieving pain and promoting healing of lower back problems such as Spinal Stenosis, Degenerative Disc Disease, Herniated Discs, Muscle Spasms, and Sciatica. The device includes an upper strap or belt worn below the breast line and a lower strap or belt worn in the normal belt position riding on the hip. At least two rigid splints extend vertically between the two belts on the left and right sides of the body. When the wearer stands or sits, the lower spine is gently decompressed.

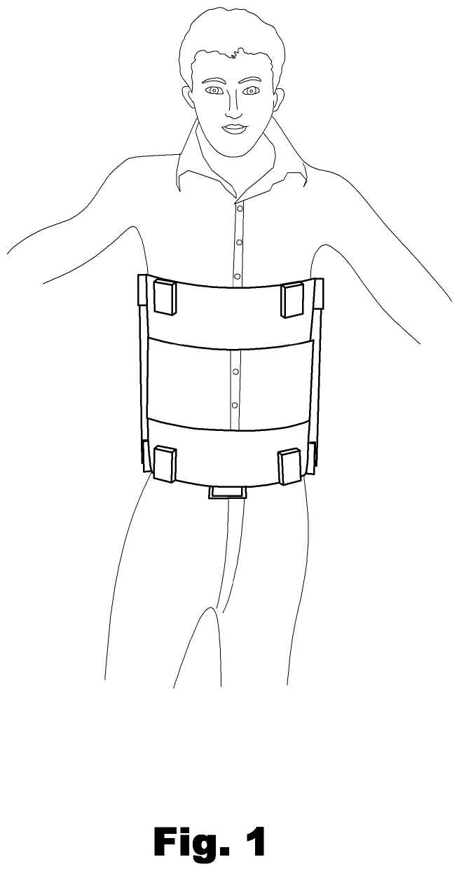

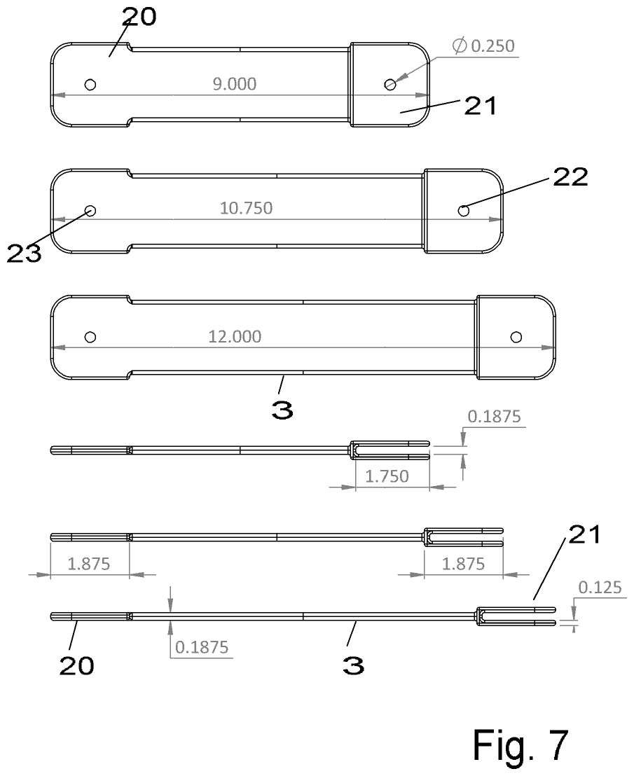

Turning to FIG. 1, an embodiment of the present invention is shown being worn. Each of the straps or belts is approximately five inches wide with sets of pockets on opposing edges. A splint can be seen extending vertically between the straps on each side of the body. The splints can be captured and held in place by the pockets. The pockets are optional; the splints can be permanently attached to the straps in some embodiments. Also, a plug or other device can be inserted into the holes at each end of the splint through the pocket and strap. The splint holes can be larger or smaller than the ¼″ pictured in FIG. 7. FIG. 1 shows the device being worn outside the clothing for clarity. While this is possible, normally the device is worn under clothing so as to not be as visible.

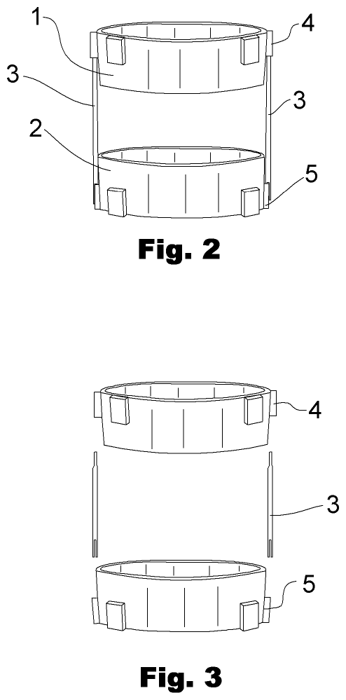

FIG. 2 shows the device assembled, but not being worn, while FIG. 3 shows it exploded. The upper and lower straps 1, 2 are approximately five inches wide. Each strap can be approximately forty-five inches long with an overlapped region that uses hook-loop fasteners known in the trade as VELCRO™. Any other method may be used to secure the straps. The straps can thus be fitted to most wearers. Longer and shorter straps can be supplied for very large and very small wearers. While, the dimensions given are preferred, the straps can be made in any convenient sizes. The preferred material for the strap is neoprene; however, any flexible material may be used, including, but not limited to leather or other polymers.

FIGS. 2 and 3 also show a pair of splints 3 on the left and right sides of the device that are received and held by pockets 4, 5. The splints can be made from any rigid material with molded rigid plastic being preferred. Other materials can be metal or wood. Any material that is rigid as a flat splint can be used. The lower end of the splints can optionally be split or U-shaped to cup over a standard belt in the case the wearer prefers to wear only an upper strap. The splints are typically between eight to thirteen inches long with preferred sizes being approximately 9.00 inches, 10.75 inches and 12.00 inches. Any length of splint may be used. The splint extends from an upper strap under the shoulders to a lower strap on the belt line. The width of the splint can be between 1 inch and 3 inches with approximately 2 inches being preferred. The width of the splint is not critical as long as the splint fits snugly against the side of the body. The thickness of the splints can be between 0.15 inches to 0.25 inches with a preferred thickness being approximately 0.1875 inches. The thickness is not critical as long as the longest splint is thick enough to not bend outward in compression (column instability), but rather remain rigid.

It should be noted in FIGS. 1-3 that the splints 3 are located directly on the sides of the user's body. They are centered laterally. Centered laterally means that they are centered on right and left sides of the torso under the armpits. The optional additional pockets are only used so that different sized users can locate a splint directly on each side of their body, i.e. center it laterally. The device will not provide the required decompression if the splints are not on the sides of the body.

The pockets 4, 5 typically have slots that receive the splints. The pockets on the upper strap are typically on its upper edge, while the pockets on the lower strap are typically on its lower edge. The two straps can be identical with the lower strap simply flipped over when worn so that its pockets are on its lower edge. The slots should match the width and thickness of the splints so that they are held firmly in place by the straps 1, 2. As stated, the pockets are optional; the splints can be permanently attached to the straps by any known method.

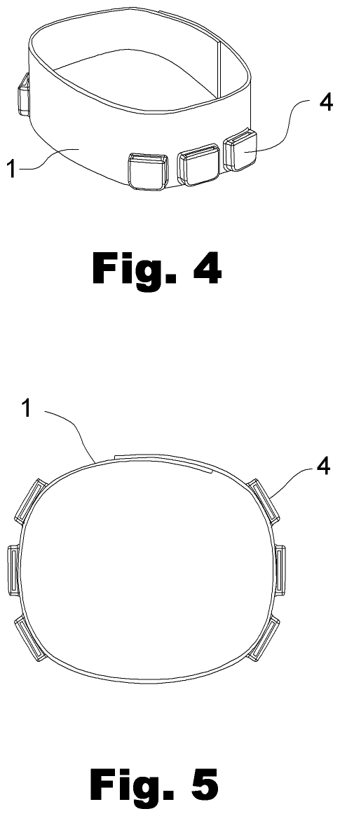

FIGS. 4 and 5 show an embodiment of the strap 1 with three pockets 4 on the left and right sides. The center pocket is used for most users. Again, the extra, optional pockets are only used to assure that the splints are aligned with the left and right side of the user's body for different sized users. While three pockets are shown on each side, any number of pockets including only one pocket (or no pockets) on each side is within the scope of the Present invention. The pockets 4 are securely attached to the strap 1. As stated, the preferred material for the strap 1 is neoprene, and the preferred material for the splint 3 is hard plastic.

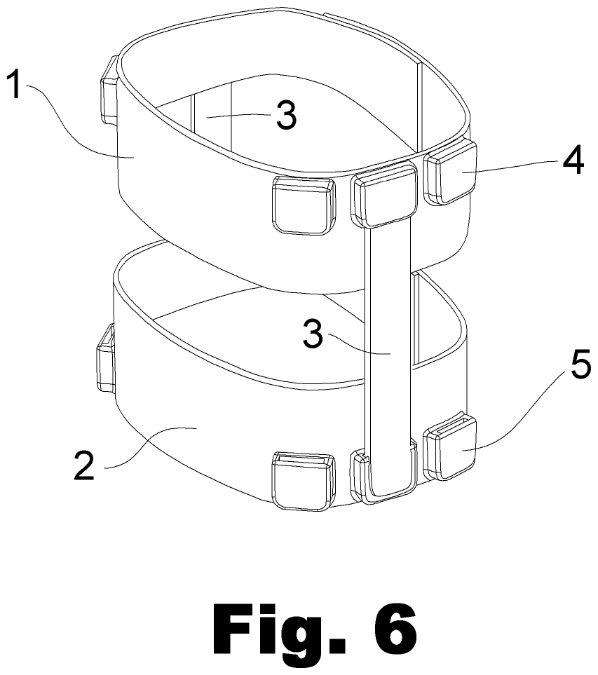

FIG. 6 shows the straps and splints of FIGS. 4-5 assembled. It should be noted that the top pockets 4 face downward, and the bottom pockets 5 face upward to receive and hold the splints. This alignment of the pockets occurs using identical straps for top and bottom by simply flipping the strap over depending upon whether it is used in the top or bottom position.

FIG. 7 shows top and side views of splints 3 of three preferred lengths 9.00 inches, 10.75 inches and 12.00 inches. Splints of these lengths can be supplied as stock items. Special splints of other sizes can also be made. Splints of any size are within the scope of the present invention. While other preferred dimensions are also shown in FIG. 7, all of the dimensions in FIG. 7 are for example only. The splints can be made with any other dimensions.

The splints 3 in FIG. 7 are shown with a flat end 20 and a forked or U-shaped end 21. The flat end 20 fits into the upper strap pocket 4, while the forked end 21 is the bottom end that either is received by the lower pocket 5 or forks around a standard belt worn around the hips. Splints can also be made with both ends flat or both ends forked.

The splints 3 of FIG. 7 are shown with small holes 23, 24 in their ends. These holes are optional, can be made any size, and can be used with removable plugs that secure the splints 3 to the pockets 4, 5. These, when used, allow the user to remove the belt and splints as a one-piece harness for convenience. However, plugs are typically not required to be used as the invention relies on the compressive forces between the straps 1, 2 to hold the splints 3 in the pockets 4, 5.

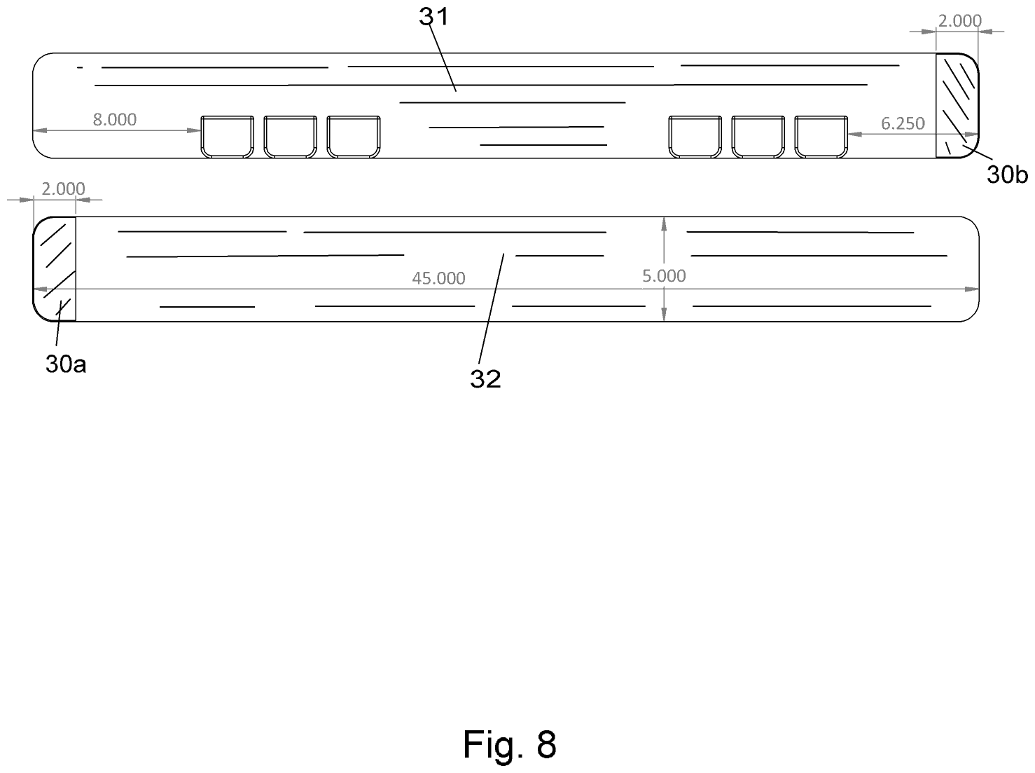

FIG. 8 shows a font and back view of a strap. FIG. 8 also shows example dimensions. Again, all of the dimensions in FIG. 8 are for example only. The straps can be made with any dimensions. The upper part of FIG. 8 shows the front of a representative strap; the lower part of FIG. 8 shows the back. Both the front and the back have a surface of loop material 31, 32. Hook material appears in strips 30a and 30b at the ends of the strap. There is hook material on both sides of the strap at different ends. This allows the strap to attach to the user's body at two different locations preventing slippage. While this is a particular embodiment of hook/loop material, any other arrangement of hook/loop material or any other method of holding the strap securely in place is within the scope of the present invention.

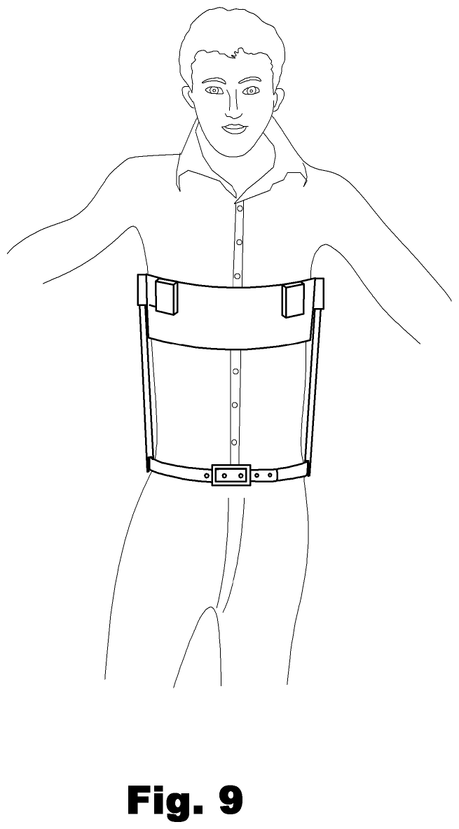

FIG. 9 shows an embodiment of the present invention being worn with only one strap using a standard belt for the lower strap. This embodiment may be preferred by users who do not wish to have to wear two straps. Again, the device is shown worn outside the user's clothing. Typically, however, the device will be worn under clothing to make it less visible.

The present invention relates to a device that decompresses the lower spine of a wearer while standing or seated relieving pain from lower back problems and providing a better environment for long-term healing. In addition to Spinal Stenosis, the present invention is useful for general Spondylosis conditions such as, degenerative disc disease, herniated disks, sciatica, muscle spasms and other causes of lower back pain.

### Improved Embodiment of the Invention (New Matter)

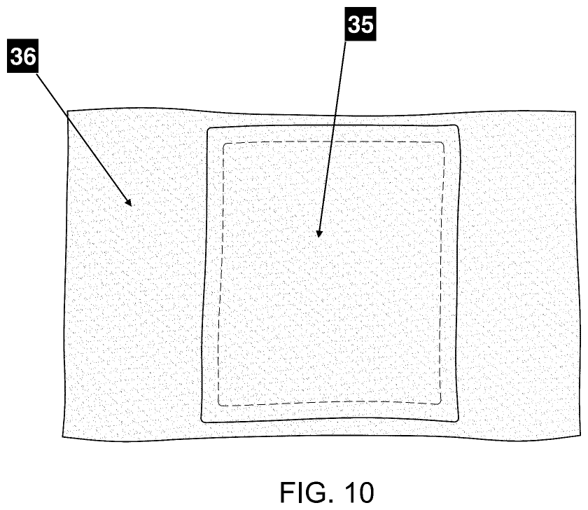

An improved embodiment of the present invention is depicted in FIGS. 10-19.

The present invention employs several pockets located on each side of neoprene bands worn around the wearer's waist and chest. These pockets are designed to hold splints that go between the upper and lower bands. The assembly creates spinal traction when the user gently bends while sitting or standing. The pockets give the wearer one option to attach the splints or bracing to the upper band, and give the wearer 3 options to anchor the bottom of the splints: (1) using the wearer's typical pants belt (2) the top rim of the wearer's pants waist band without a belt, or (3) placing the splint into one of 3 pockets located along each side of the lower neoprene band. Choosing different pockets for insertion of the splints gives the wearer the ability to move the splint ends enabling different comfort levels and gives the wearer the option of adjusting the forward and backward angles of the splints depending on the user's pocket in the upper and lower bands.

Adjusting the angles of the splints by moving them horizontally changes the amount of upper body weight resistance and the pressure points where the upper body weight rests. For instance, if the top of the braces or struts point forward more than the bottom of the braces, the wearer would feel more decompression of the lower spine when leaning backward as his/her upper body weight shifts over the back of the top belt. Or the wearer could adjust the splints to their lateral vertical positions, which would allow greater spinal decompression when leaning forward, but less when leaning backward. In addition to providing the user selective levels and points of decompression, changing the splint angles in this way allows the wearer to find the most comfortable position so that the splint ends are not irritating bony areas of the pelvis or ribs. The greater the wearer's flexibility to horizontally move the pockets and thus the splints, the greater the amount of comfort, convenience, and type of decompression intensity and pain relief. The present invention, however, had a limited number of pockets to choose from to change the splint angles. This horizontal movement feature, among others, allows the upper and lower splints to be adjusted horizontally by the user.

The new embodiment of FIG. 10 which provides a pocket made of hook and loop material at 35, sewn or affixed to the top of a flat moveable hook and loop material piece at 36, FIG. 10, now allows the wearer an infinite number of options to adjust both splints horizontally by simply pulling off and moving the pocket assembly and placing it anywhere along each band instead of being restricted to using one of the stationary pocket choices in the present invention. In the new embodiment of FIGS. 10, 35 and 36 together are referred to as a “pocket assembly”. The new embodiment pocket assembly is light weight, made entirely of loop material on the outside and hook material on the inside but could be made of any light weight and adhering materials. The new pocket assembly embodiment in FIG. 10 also allows the wearer to simply pull off the pocket assembly from the FIGS. 6, 1 and 2 belts conveniently with the splint still inserted and to move it anywhere horizontally along the face of either belt instead of placing the splint only in the pre-determined pockets in the original invention. This new embodiment provides the wearer with more ways to achieve comfort, different levels of decompression and choices of pressure point locations.

In the new embodiment placing the pockets anywhere along the horizontal bands allows the user to place the splints at a different angle on the left side of the body than on the right side, further accommodating wearers that may have more pain on one side of the spine than the other.

The present invention used the method of transferring weight away from the lower spine. In the present invention FIG. 6, 1 grasps much of the weight of the wearer's upper body. The force of this weight then rests on the top of the splint (“A”), then moves through the splint (“B”) to the lower band pocket, waistline or wearer's own belt where it then rests on (“D”), the waist or hips point where the splint ends. The weight, which has moved through the splint to point (“D”) at the bottom of the system, has thus bypassed the wearer's mid to lower back (“C”). This offloading of upper bodyweight from “A” through “B”, bypassing “C” and resting on “D” allows the compressed area of the lower back to gently expand, allowing delicate compressed disc padding between the vertebrae to “decompress”, rehydrate and puff up again and regain their height and padding effect.

The present invention employed several sizes of fixed length splints through which the weight of the upper body would pass to the hips creating the comfort of actual decompression of the lower spine and to accommodate various people of different height. Although different sized splints are employed in the present invention, for more effective results they required occasional adjustment of the entire straps and splint assembly by the wearer in order to create maximum decompression. This was accomplished by pulling or pushing downward on the whole straps and splint assembly before sitting.

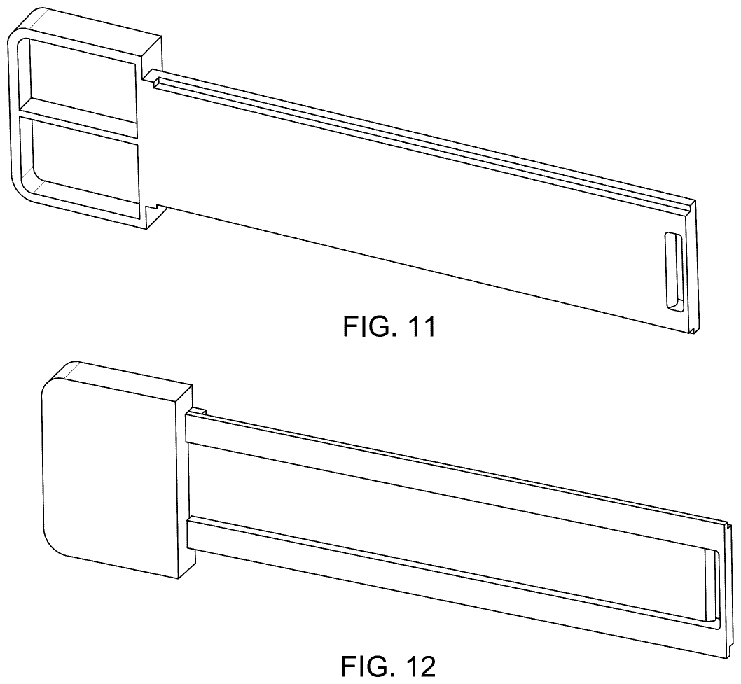

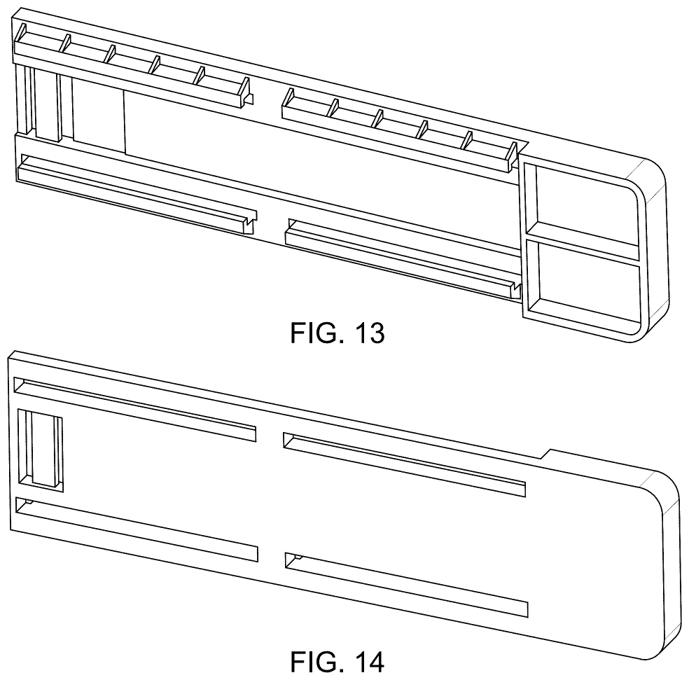

The fixed length splints in the present invention are also not as convenient or useful for accommodating wearers that are extremely tall or short. The new embodiments shown in FIGS. 11,12,13,14, 15, 16 and 17 illustrate a unique reverse sliding mechanism that accommodates wearers up to 6′ 10″ tall. FIG. 11 illustrates the outside of one half of the new sliding splint mechanism. FIG. 12 illustrates the other side of that same piece. FIGS. 13 and 14 illustrate each side of the other half of the sliding splint mechanism. FIG. 13 shows the rails into which the new embodiment splint illustrated in FIGS. 11 and 12 slides.

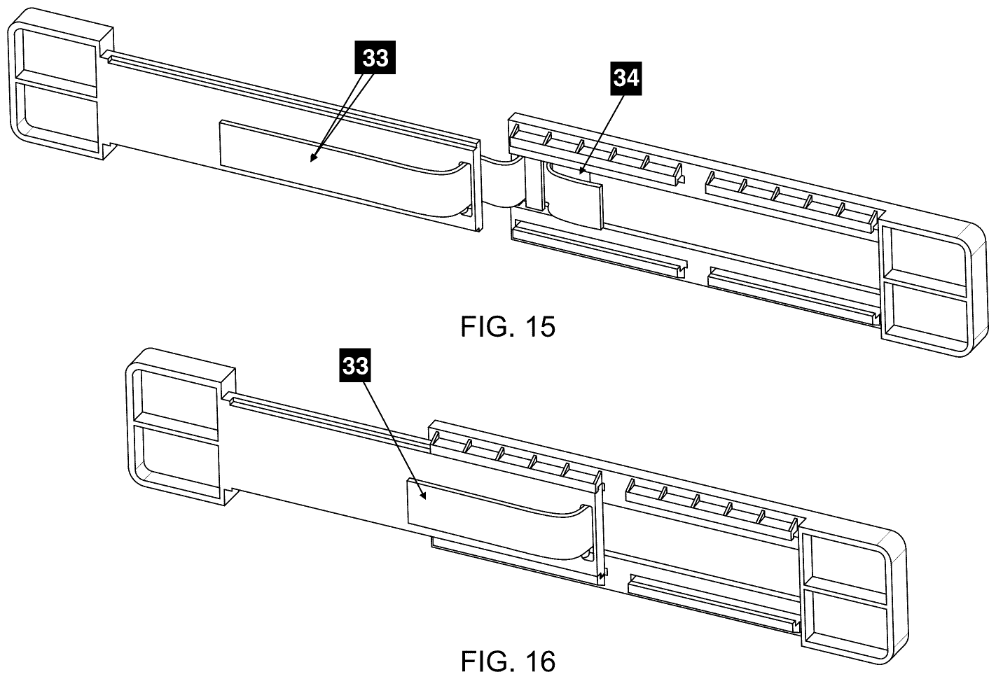

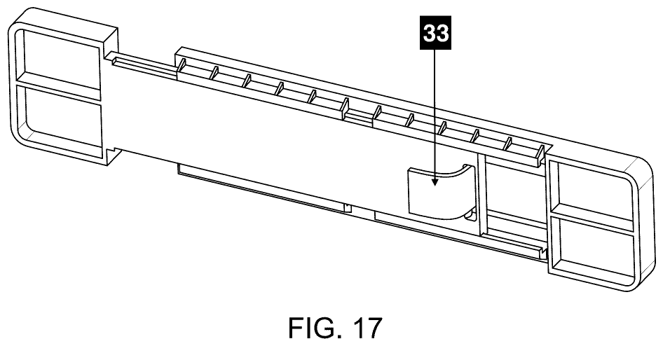

FIG. 15 at 34, illustrates the location where the hook and loop pull strap connects to the FIG. 13. splint half by glue, rivet or other means of fastening. FIG. 16 at 33 illustrates the completed splint assembly of FIGS. 11-14 and illustrates the location where the hook and loop pull strap emerges from the FIGS. 11 and 12 splint half when it is inserted into the FIG. 13 rails. FIG. 17 at 33 illustrates the completed splint when shortened and part of the hook loop strap is pulled inside the splint assembly. And FIG. 16 at 33 illustrates the completed splint when lengthened and part of the hook loop strap is hanging outside of the splint assembly.

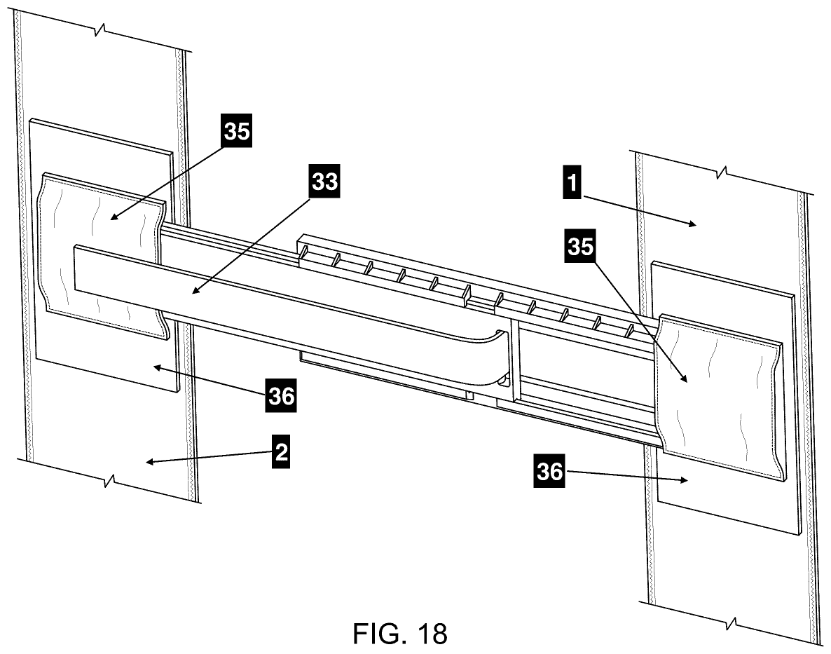

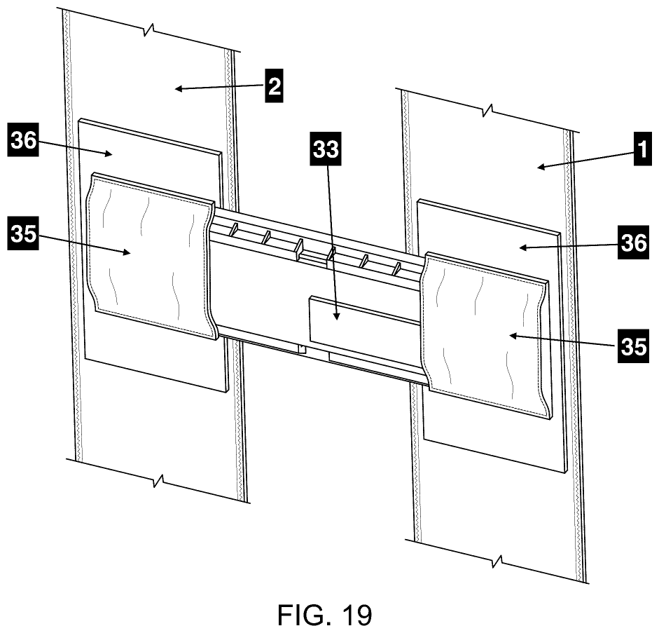

When the hook and loop materials are inserted into the splint halves, the FIGS. 11 and 12 splint half slides into the FIGS. 13 and 14 splint half and the entire splint assembly is inserted into the 35 and 36 pocket assembly in FIGS. 18 and 19. FIG. 19 at 33 illustrates the completed splint when shortened and part of the hook loop strap is pulled inside the splint assembly. And FIG. 18 at 33 illustrates the completed splint when lengthened and part of the hook loop strap is stuck to the pocket outside of the splint assembly.

This entire new embodiment splint assembly is placed into the new embodiment end pocket and pocket assembly at 35 and 36 and the completed splint and pocket assemblies are attached by hook and loop material to the FIG. 1 belts at FIGS. 18 and 19. When all parts are assembled and attached to the 1 belt, the whole straps and splint assembly allows the wearer to simply pull out the convenient hook and loop tape located within the new splint in 33 FIG. 18, which extends the height of the splint for taller people, or by letting out the hook and loop pull tape to shorten the splint for shorter wearers, at 33 FIG. 19.

The remaining 33 hook and loop tape, whether short or long, then sticks to the hook and loop pocket securing the splints at their wearer chosen height, preventing excess hook and loop pull at 35 FIG. 18. This new user determined adjustability firmly adjusts the device to the user's size to help decompress the lower spine, prevents inadvertent shortening of the splints and increases a user's comfort when wearing the device for extended periods of time.

Although the present invention provides effective spinal decompression, the improved embodiments eliminate these inconveniences, providing greater comfort and greater spinal decompression by employing a splint mechanism that no longer requires the wearer to stand upright and interrupt what the wearer is doing in order to adjust the straps and splints assembly to provide greater decompression. The new embodiment, because of its increased height and lateral movement also improves a condition in the elderly known as kyphosis. Kyphosis, which is a normal part of the aging process, compresses the total length of the spine causing a person to tilt forward while walking. The illustrated new embodiments assist the elderly to straighten when walking by creating more walking angles to decompress the lower spine, relieving pain and also acting as a physical reminder to remain erect and not slouch.

The improved embodiments, by employing uniquely adjustable splints, enables the user to have the convenience of using different comfort and bilateral decompression options without having to stand up to adjust the whole assembly. The improved embodiments, take the place of several sets of splint sizes, are lighter weight, accommodate shorter and taller people than the present design, and eliminate the need to ship several different sized splints to the user. The updated adjustable “reverse pull” mechanism and design of the adjustable brace is therefore more effective and provides more convenience choices to the wearer.

The lower splint of the improved embodiments could employ the “U” shaped splint ends of the present invention for connecting the wearer's pants rim or the user's own belt to the bottom splints, but the updated design is more convenient, as the user is no longer restricted to using a stationary pocket and can adjust the length of the splints by pulling or letting out the hook and loop pull, one end of which is firmly affixed at 34 FIG. 15 via rivet, glue or by other means to the inside of the splint. Moreover, the movable one-piece pocket assembly FIG. 10, provides the user the choice to place the pockets anywhere around the outside of the band, helping additional “walk leaning” conditions such as kyphosis. The adjustable length splints in the improved embodiment now allow a user to change length and increase compression without having to get up from a chair. When the pocket assembly hook surface is stuck to the loop waist or chest band with the splint end in its pocket, the user can simply pull down or let out the hook and loop pull while sitting.

After the desired splint height is selected, the hook side of the 33 pull then sticks to the outside of the loop material of the pocket assembly in FIG. 18 securing the splint at the selected length from collapsing. When the splint is completely compressed, most of the pull hides under the pocket FIG. 19. The pull can be used to adjust the splint short or long to precisely fit the wearer. These improved embodiments also insure after adjustment that the hook and loop pull lies flat against the device and out of the way of the wearer, FIG. 33.

Unlike other inventions, with its low cost and narrow profile, the new comfort and decompression options, the improved embodiments of the device with its reverse pull mechanism, is lighter weight and more unique than the present invention. The device can be shipped at lower cost, making it more affordable to the user. Moreover, the improved embodiment, which is superior to those of the predecessor in portability, comfort and convenience, as well as to any existing wearable decompression devices in the marketplace, also addresses more therapeutic issues.

Early trials indicate the product may have outstanding commercial success and will improve the lives of the average person who suffers from back pain. Its fully adjustable options, lightweight and portable design, unlike that of the prior art, allow it to be worn inconspicuously under clothing comfortably all day, and it is easily washable and packable. The present embodiment provides more convenient and better therapeutic options to the user and causes less impact on the user's daily activities than any previous technique or device.

### I. Original Invention—May Retain Parent Priority

I claim:

Claim 1. A method of relieving lower back pain using only a wearer's body weight comprising:
providing a first flexible strap encircling a patient's upper torso;
providing a second flexible strap encircling the patient's belt-line;
providing a first continuous, rigid splint along a left side of the patient's body from the first flexible strap to the second flexible strap;
providing a second continuous, rigid splint along a right side of the patient's body from the first flexible strap to the second flexible strap allowing weight of the wearer's upper body, shoulders and head to press downward on the first and second splints displacing the weight of the upper body to the hips relieving pressure on the lower back and creating an upward reactive force on the patient's lower spine stretching lower spine upward relieving pressure on the patient's lower vertebrae relieving lower back pain.

Claim 2. The method of claim 1 wherein the second flexible strap is a wearer's belt.

Claim 3. The method of claim 1 wherein lower ends of the first and second continuous rigid splints are received in and secured in pockets attached to the second flexible strap allowing the splints to move without pulling out of the pockets.

Claim 4. The method of claim 3 wherein the continuous rigid splints are secured in the pockets with Velcro (TM) straps.

Claim 5. The method of claim 1 wherein upper ends of the first and second continuous rigid splints are received in pockets attached to the first flexible strap, the ends being secured on one side of the splint to inside of the pocket allowing the splints to rock forward, backward or sideways without pulling out of the pockets.

Claim 6. The method of claim 5 wherein the continuous rigid splints are secured in the pockets with Velcro (TM) straps.

Claim 7. the method of claim 1 wherein the splints can be removed and the splints and straps rolled up as one piece when not in use.

Claim 8. The method of claim 1, wherein lower ends of the first and second continuous rigid splints are attached to a wearers belt using a “U” shaped end on each splint.

Claim 9. The method of claim 1 wherein the first and second flexible straps and the first and second continuous rigid splints are worn under the clothing.

Claim 10. A method of relieving lower back pain using only a person's body weight comprising:
encircling a patient's upper torso with a first flexible strap;
encircling the patient's belt-line with a second flexible strap;
placing a first continuous, rigid splint along a left side of the patient's body from the first flexible strap to the second flexible strap;
placing a second continuous, rigid splint along a right side of the patient's body from the first flexible strap to the second flexible strap allowing weight of the wearer's upper body, shoulders and head to press downward on the first and second splints displacing the weight of the upper body to the hips relieving pressure on the lower back and creating an upward reactive force on the patient's lower spine stretching lower spine upward relieving pressure on the patient's lower vertebrae relieving lower back pain.

Claim 11. The method of claim 10 wherein the second flexible strap is a wearer's belt.

Claim 12. The method of claim 10 wherein lower ends of the first and second continuous rigid splints are received in and secured in pockets attached to the first and second continuous rigid splints and worn under the clothing and to the second flexible strap allowing the splints to move without pulling out of the pockets.

Claim 13. The method of claim 12 wherein the continuous rigid splints are secured in the pockets with Velcro™ straps.

Claim 14. The method of claim 10 wherein upper ends of the first and second continuous rigid splints are received in pockets attached to the first flexible strap, the ends being secured on one side of the splint to inside of the pocket allowing the splints to rock forward, backward or sideways without pulling out of the pockets.

Claim 15. The method of claim 14 wherein the continuous rigid splints are secured in the pockets with Velcro™ straps.

Claim 16, The method of claim 10 wherein the splints can be removed and the splints and straps rolled up as one piece when not in use.

Claim 17 The method of claim 10, wherein lower ends of the first and second continuous rigid splints are attached to a wearers belt using a “U” shaped end on each splint.

Claim 18. The method of claim 10 wherein the first and second flexible straps and second continuous rigid splints are worn under the clothing.

### II. New Matter—Adjustable Splints and Moveable Pockets (CIP Priority)

Claim 19. A wearable spinal decompression device comprising:
an upper flexible strap configured to encircle a wearer's upper torso;
a lower flexible strap configured to encircle the wearer's waist;
at least one continuous, adjustable-length splint extending vertically between the upper and lower straps;
and at least one horizontally moveable pocket attached to at least one of the straps using hook-and-loop fasteners, the pocket being configured to receive and retain an end of the splint.

Claim 20. The device of claim 19, wherein each splint includes a reverse-pull adjustable length mechanism secured by a hook-and-loop pull tape.

Claim 21. The device of claim 19, wherein the splints can be adjusted in length without removing the device from the body.

Claim 22. The device of claim 19, wherein each splint is removably retained in an independently horizontally movable pocket secured along the exterior surface of the strap.

Claim 23. The device of claim 19, wherein the splints can be set at differing lateral positions on the left and right sides to adjust decompression angles.

Claim 24. The device of claim 19, wherein the straps and splints are configured to be worn under clothing.

### III. New Method Claims—Adjustable Features

Claim 25. A method of relieving lower back pain using a wearable decompression device, comprising:
placing an upper strap around a wearer's torso below the chest;
placing a lower strap around the waist or belt-line;
inserting first and second adjustable-length splints vertically between the upper and lower straps on each side of the torso;
securing each end of the splints into horizontally movable pockets attached to the straps using hook-and-loop fasteners;
adjusting the vertical length and angle of the splints to redirect the upper body weight toward the hips, thereby decompressing the lower spine.

Claim 26. The method of claim 25, wherein the adjustable-length splints are modified using a reverse-pull mechanism.

Claim 27. The method of claim 25, wherein each splint is adjusted independently to accommodate user height or spinal asymmetry.

Claim 28. The method of claim 25, wherein the pockets and splints remain in place while the straps are rolled up for storage.

### Abstract

A device that decompresses the lower spine while the wearer is seated or standing. The device includes two belt-shaped straps. The first strap circles the upper torso just below the breast line. The second strap circles the waist at or slightly above the normal belt line riding on the hip. In the present invention, the lower strap can be a normal belt. The straps can be equipped with pockets that are aligned along the upper edge of the upper strap and the lower edge of the lower strap designed to receive and hold flat splints. Typically, two rigid flat splints extend vertically between the straps centered laterally on the left and right sides of the torso. When the device is worn, it decompresses the lower spine when the person stands or sits. In the new embodiments the flat splints are adjustable length and the pockets are horizontally moveable along the lower and upper straps and the “U”shaped end that attaches to the wearers belt is optional.

1-18. (canceled)

19. A wearable spinal decompression device comprising:
an upper flexible strap configured to encircle a wearer's upper torso;
a lower flexible strap configured to encircle the wearer's waist;
at least one continuous, adjustable-length splint extending vertically between the upper and lower straps;
and at least one horizontally moveable pocket attached to at least one of the straps using hook-and-loop fasteners, the pocket being configured to receive and retain an end of the splint.

20. The device of claim 19, wherein each splint includes a reverse-pull adjustable length mechanism secured by a hook-and-loop pull tape.

21. The device of claim 19, wherein the splints can be adjusted in length without removing the device from the body.

22. The device of claim 19, wherein each splint is removably retained in an independently horizontally movable pocket secured along the exterior surface of the strap.

23. The device of claim 19, wherein the splints can be set at differing lateral positions on the left and right sides to adjust decompression angles.

24. The device of claim 19, wherein the straps and splints are configured to be worn under clothing.

25. A method of relieving lower back pain using a wearable decompression device, comprising:
placing an upper strap around a wearer's torso below the chest;
placing a lower strap around the waist or belt-line;
inserting first and second adjustable-length splints vertically between the upper and lower straps on each side of the torso;
securing each end of the splints into horizontally movable pockets attached to the straps using hook-and-loop fasteners;
adjusting the vertical length and angle of the splints to redirect the upper body weight toward the hips, thereby decompressing the lower spine.

26. The method of claim 25, wherein the adjustable-length splints are modified using a reverse-pull mechanism.

27. The method of claim 25, wherein each splint is adjusted independently to accommodate user height or spinal asymmetry.

28. The method of claim 25, wherein the pockets and splints remain in place while the straps are rolled up for storage.
